(19) United States
(12) Patent Application Publication
(10) Pub. No.: US 2025/0311988 A1
(43) Pub. Date: Oct. 9, 2025

(54) X-RAY CT APPARATUS

(71) Applicant: CANON MEDICAL SYSTEMS CORPORATION, Tochigi (JP)

(72) Inventors: Shota ISHIKAWA, Otawara (JP); Hiroaki MIYAZAKI, Otawara (JP)

(73) Assignee: CANON MEDICAL SYSTEMS CORPORATION, Tochigi (JP)

(21) Appl. No.: 19/098,012

(22) Filed: Apr. 2, 2025

(30) Foreign Application Priority Data
Apr. 3, 2024 (JP) 2024-059992

(51) Int. Cl. A61B 6/03; A61B 6/00; A61B 6/42

(52) U.S. Cl. CPC A61B 6/035; A61B 6/4241; A61B 6/5205

(57) ABSTRACT

An X-ray computed tomography apparatus according to an exemplary embodiment includes an X-ray detector, a data acquisition unit, a rotating portion, a 1st processing circuitry, and a 2nd processing circuitry. The X-ray detector is configured to output output data corresponding to energy of an X-ray photon. The data acquisition unit is configured to acquire the output data from the X-ray detector and output count data for each of a plurality of energy bins. The rotating portion is configured to rotatably support the X-ray detector and the data acquisition unit. The 1st processing circuitry is provided in the rotating portion and is configured to generate data related to a plurality of material compositions based on the count data and output the data related to the plurality of material compositions. The 2nd processing circuitry is configured to perform a reconstruction process based on the data related to the plurality of material compositions.

### CROSS-REFERENCE TO RELATED APPLICATIONS

This application is based upon and claims the benefit of priority from Japanese Patent Application No. 2024-059992, filed on Apr. 3, 2024; the entire contents of all of which are incorporated herein by reference.

### FIELD

Exemplary embodiments described herein relate generally to an X-ray computed tomography (CT) apparatus.

### BACKGROUND

In a photon counting X-ray computed tomography (CT) apparatus, an X-ray detector counts the number of incident X-ray photons within set energy ranges using counters referred to as energy bins. The counted data is transmitted from a detector unit of a rotating portion to a fixed portion, and preprocessing and image reconstruction of the data are performed as needed in a console.

Increasing the number of the energy bins improves accuracy of energy information, which is a feature of the photon counting X-ray CT apparatus. However, as the number of the energy bins increases, it becomes difficult to transmit all the data in real time due to constraints on the amount of data in data transmission from the rotating portion of a gantry to the fixed portion.

### BRIEF DESCRIPTION OF THE DRAWINGS

FIG. 1 is a diagram illustrating an example of a configuration of an X-ray computed tomography (CT) apparatus according to a first exemplary embodiment;

FIG. 2 is a diagram illustrating a configuration of a rotating portion of the X-ray CT apparatus according to the first exemplary embodiment;

FIG. 3 is a flowchart illustrating a flow of processing performed by the X-ray CT apparatus according to the first exemplary embodiment;

FIG. 4 is a diagram illustrating the processing according to the first exemplary embodiment;

FIG. 5 is a flowchart illustrating a process in step S300 according to the first exemplary embodiment in more detail;

FIG. 6 is a flowchart illustrating a process in step S300 according to a third exemplary embodiment in more detail;

FIG. 7 is a diagram illustrating processing according to the third exemplary embodiment;

FIG. 8 is a diagram illustrating processing according to a fourth exemplary embodiment; and

FIG. 9 is a flowchart illustrating a process in step S300 according to the fourth exemplary embodiment in more detail.

### DETAILED DESCRIPTION

An X-ray computed tomography apparatus according to an exemplary embodiment includes an X-ray detector, a data acquisition unit, a rotating portion, a 1st processing circuitry, and a 2nd processing circuitry. The X-ray detector is configured to output output data corresponding to energy of an X-ray photon. The data acquisition unit is configured to acquire the output data from the X-ray detector and output count data for each of a plurality of energy bins. The rotating portion is configured to rotatably support the X-ray detector and the data acquisition unit. The 1st processing circuitry is provided in the rotating portion and is configured to generate data related to a plurality of material compositions based on the count data and output the data related to the plurality of material compositions. The 2nd processing circuitry is configured to perform a reconstruction process based on the data related to the plurality of material compositions.

Various embodiments will be described hereinafter with reference to the accompanying drawings.

### First Exemplary Embodiment

Exemplary embodiments of X-ray CT apparatuses will be described in detail below with reference to the drawings.

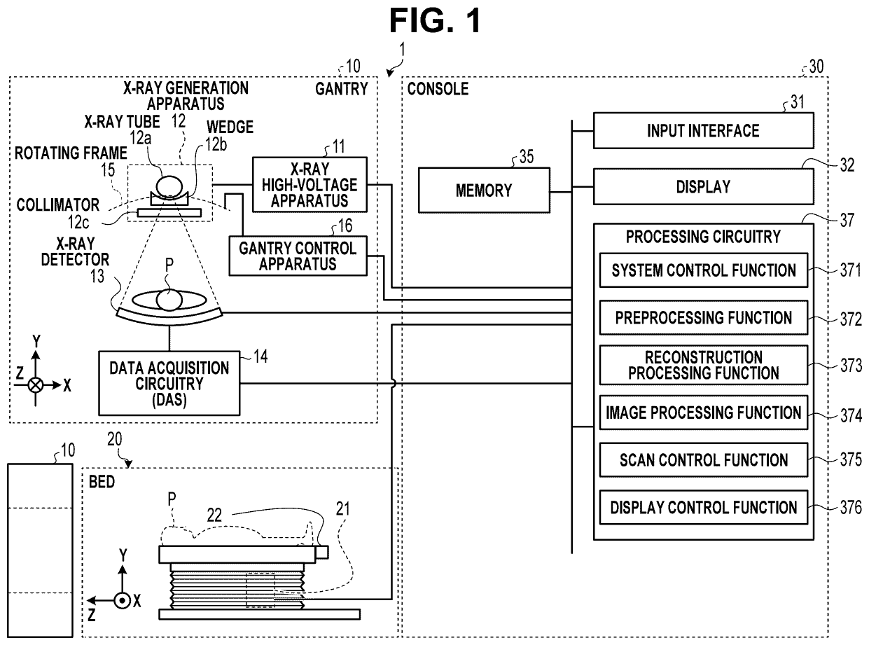

FIG. 1 is a diagram illustrating an example of a configuration of an X-ray CT apparatus 1 according to a first exemplary embodiment. The X-ray CT apparatus 1 is a photon counting CT apparatus. Specifically, the X-ray CT apparatus 1 is an apparatus capable of reconstructing X-ray CT image data with a high signal-to-noise (S/N) ratio by counting X-ray photons that have passed through a subject P using a photon counting detector using a photon counting method, rather than a conventional integrating detector (of a current mode measurement method). As illustrated in FIG. 1, the X-ray CT apparatus 1 according to the first exemplary embodiment includes a gantry 10, a bed 20, and a console 30.

The gantry 10 is an apparatus that irradiates the subject P with X-rays and acquires data related to the X-rays that have passed through the subject P. The gantry 10 includes an X-ray high-voltage apparatus 11, an X-ray generation apparatus 12, an X-ray detector 13, data acquisition circuitry 14, a rotating frame 15, and a gantry control apparatus 16. Further, an orthogonal coordinate system consisting of X, Y, and Z axes is defined in the gantry 10 as illustrated in FIG. 1. Specifically, the X-axis represents a horizontal direction, the Y-axis represents a vertical direction, and the Z-axis represents a rotation axis direction of the rotating frame 15 when the gantry 10 is in a non-tilted state.

The rotating frame 15 is an annular frame that supports the X-ray generation apparatus 12 and the X-ray detector 13 so that the X-ray generation apparatus 12 and the X-ray detector 13 face each other across the subject P, and is rotated at high speed on a circular path centered on the subject P by the gantry control apparatus 16.

The X-ray generation apparatus 12 is an apparatus that generates X-rays and irradiates the subject P with the generated X-rays. The X-ray generation apparatus 12 includes an X-ray tube 12a, a wedge 12b, and a collimator 12c.

The X-ray tube 12a is a vacuum tube that receives a high voltage supplied from the X-ray high-voltage apparatus 11 and emits thermal electrons from a cathode (sometimes referred to as “filament”) toward an anode (target). The X-ray tube 12a irradiates the subject P with an X-ray beam as the rotating frame 15 rotates. Specifically, the X-ray tube 12a generates an X-ray using the high voltage supplied from the X-ray high-voltage apparatus 11.

Further, the X-ray tube 12a generates an X-ray beam that spreads out at a fan angle and a cone angle. For example, the X-ray tube 12a can continuously emit X-rays around the entire periphery of the subject P for full reconstruction, or can continuously emit X-rays in an exposure range (180 degrees+fan angle) that enables half reconstruction for the half reconstruction, under control of the X-ray high-voltage apparatus 11. Further, the X-ray tube 12a can expose X-rays (pulse X-rays) intermittently at a preset position (tube position) under control of the X-ray high-voltage apparatus 11.

The wedge 12b is an X-ray filter for adjusting an X-ray dose of X-rays emitted from the X-ray tube 12a. Specifically, the wedge 12b is a filter that transmits and attenuates X-rays emitted from the X-ray tube 12a so that the X-rays emitted from the X-ray tube 12a toward the subject P have a predefined distribution. For example, the wedge 12b is a filter made by processing aluminum to a predetermined target angle and thickness. A wedge is also referred to as a wedge filter or a bow-tie filter.

The collimator 12c includes a lead plate and has a slit in a portion thereof. For example, the collimator 12c uses the slit to narrow an irradiation range of the X-rays, whose X-ray dose has been adjusted by the wedge 12b, under control of the X-ray high-voltage apparatus 11 described below.

The X-ray source of the X-ray generation apparatus 12 is not limited to the X-ray tube 12a. For example, the X-ray generation apparatus 12 may include, in place of the X-ray tube 12a, a focus coil that focuses an electron beam generated by an electron gun, a deflection coil that electromagnetically deflects the electron beam, and a target ring that surrounds half of the periphery of the subject P and generates X-rays when the deflected electron beam collides with the target ring.

The X-ray high-voltage apparatus 11 includes a high-voltage generation apparatus and an X-ray control apparatus. The high-voltage generation apparatus includes electrical circuitry such as a transformer and a rectifier, and has a function to generate the high voltage applied to the X-ray tube 12a. The X-ray control apparatus controls an output voltage based on the X-ray emitted by the X-ray tube 12a. The high-voltage generation apparatus may employ a transformer or inverter method. For example, the X-ray high-voltage apparatus 11 adjusts the X-ray dose emitted toward the subject P by adjusting a tube voltage and a tube current supplied to the X-ray tube 12a. Further, the X-ray high-voltage apparatus 11 is controlled by processing circuitry 37 of the console 30.

The gantry control apparatus 16 includes processing circuitry, which includes a central processing unit (CPU), and a drive mechanism such as a motor and an actuator. The gantry control apparatus 16 has a function of controlling operation of the gantry 10 upon receiving an input signal from an input interface 31 attached to the console 30 or an input interface attached to the gantry 10. For example, the gantry control apparatus 16 rotates the rotating frame 15 upon receiving the input signal to control the X-ray tube 12a and the X-ray detector 13 to rotate on a circular path centered on the subject P, control the gantry 10 to tilt, and control the bed 20 and a top board 22 to operate. The gantry control apparatus 16 is controlled by the processing circuitry 37 of the console 30.

The X-ray detector 13 is an example of a photon counting detector that includes a plurality of detection elements and outputs a signal corresponding to the counted number of X-ray photons. The X-ray detector 13 includes, for example, a plurality of rows of X-ray detection elements (also referred to as “sensors” or simply as “detection elements”), and in each of the rows, a plurality of X-ray detection elements is arranged in a channel direction along a single arc centered on a focal point of the X-ray tube 12a. The X-ray detector 13 has a structure in which the plurality of rows of X-ray detection elements, the plurality of X-ray detection elements being arranged in the channel direction in each of the rows, is arranged in a slice direction. Each of the X-ray detection elements of the X-ray detector 13 detects an X-ray that has been emitted from the X-ray generation apparatus 12 and has passed through the subject P and outputs an electrical signal (pulse) corresponding to the X-ray dose. The electrical signal output by each of the X-ray detection elements is also referred to as a detection signal. The data acquisition circuitry 14 (Data Acquisition System (DAS)) is circuitry that acquires a counting processing result from each of the detection elements of the X-ray detector 13 and generates detection data (projection data). In other words, the data acquisition circuitry 14 acquires counting results from the X-ray detector 13. The detection data herein is, for example, a sinogram. The data acquisition circuitry 14 is an example of a data acquisition unit.

The console 30 is an apparatus that receives an operation on the X-ray CT apparatus 1 by an operator and reconstructs X-ray CT image data using the sinogram (counting results) acquired by the gantry 10. The console 30 includes the input interface 31, a display 32, a memory 35, and the processing circuitry 37, as illustrated in FIG. 1.

The input interface 31 receives various input operations from the operator, converts the received input operations into electrical signals, and outputs the electrical signals to the processing circuitry 37. For example, the input interface 31 receives, from the operator, an acquisition condition for acquiring projection data, a reconstruction condition for reconstructing X-ray CT image data, and an image processing condition for generating image data (postprocessing image data) from the X-ray CT image data. The input interface 31 is implemented using, for example, a mouse, a keyboard, a trackball, a switch, a button, and/or a joystick.

The display 32 displays various types of information. For example, the display 32 outputs an image (X-ray CT image) based on image data generated by the processing circuitry 37 and a graphical user interface (GUI) for receiving various operations from the operator. The display 32 includes, for example, a liquid crystal display or a cathode ray tube (CRT) display.

The memory 35 is implemented using, for example, a semiconductor memory device such as a random access memory (RAM) and a flash memory, a hard disk, or an optical disk. The memory 35 stores, for example, projection data and reconstruction image data. The memory 35 is an example of a storage unit.

The processing circuitry 37 executes, for example, a system control function 371, a preprocessing function 372, a reconstruction processing function 373, an image processing function 374, a scan control function 375, and a display control function 376. For example, the processing functions executed by the system control function 371, the preprocessing function 372, the reconstruction processing function 373, the image processing function 374, the scan control function 375, and the display control function 376, which are components of the processing circuitry 37 illustrated in FIG. 1, are recorded in forms of computer-executable programs in the memory 35. The processing circuitry 37 is implemented using, for example, a processor. The processing circuitry 37 implements a function by reading a program corresponding to the function from the memory 35 and executing the read program. In other words, the processing circuitry 37 having read the programs has the functions illustrated in the processing circuitry 37 in FIG. 1. The system control function 371, the preprocessing function 372, the reconstruction processing function 373, the image processing function 374, the scan control function 375, and the display control function 376 are examples of a system control unit, a preprocessing unit, a reconstruction processing unit, an image processing unit, a scan control unit, and a display control unit, respectively. The processing circuitry 37 is an example of a 2nd processing circuitry.

The system control function 371 controls the various functions of the processing circuitry 37 based on the input operations received from the operator via the input interface 31.

The preprocessing function 372 generates raw data by performing preprocessing, such as logarithmic transformation processing, offset correction processing, inter-channel sensitivity correction processing, inter-channel gain correction processing, pile-up correction processing, response function correction processing, and beam hardening correction, on the detection data output from the data acquisition circuitry 14. Then, the preprocessing function 372 stores the raw data in the memory 35.

The reconstruction processing function 373 generates X-ray CT image data by performing reconstruction processing using a method such as the filtered back projection method and the iterative reconstruction method on the raw data generated by the preprocessing function 372. The reconstruction processing function 373 stores the reconstructed X-ray CT image data in the memory 35.

The projection data generated from the sinogram (counting result) acquired through photon counting CT includes information about energy of X-rays attenuated as a result of passing through the subject P. Thus, the reconstruction processing function 373 can reconstruct, for example, X-ray CT image data for a specific energy component. The reconstruction processing function 373 can also reconstruct, for example, X-ray CT image data for each of a plurality of energy components.

The reconstruction processing function 373 generates, for example, image data in which color tones corresponding to different energy components are assigned to pixels of the X-ray CT image data for the energy components and a plurality of pieces of X-ray CT image data that is color-coded based on the energy components is superimposed. Further, the reconstruction processing function 373 can generate, for example, image data that enables identification of a material using a K absorption edge specific to the material. Other types of image data generated by the reconstruction processing function 373 include monochromatic X-ray image data, density image data, and effective atomic number image data.

There is a technique for discriminating the type, abundance, and density of each material contained in the subject P based on differing X-ray absorption characteristics of materials, as an application of X-ray CT. The technique is referred to as material discrimination. For example, the reconstruction processing function 373 performs the material discrimination on the projection data and acquires material discrimination information. Then, the reconstruction processing function 373 reconstructs material discrimination image data representing a material discrimination image using the material discrimination information, which is a result of the material discrimination.

The image processing function 374 converts the X-ray CT image data generated by the reconstruction processing function 373 into various types of image data, such as tomographic image data of a cross-section and three-dimensional image data obtained through rendering processing, based on the input operations received from the operator via the input interface 31 and using a publicly-known method. The image processing function 374 stores the various types of converted image data in the memory 35.

The scan control function 375 controls a CT scan performed on the gantry 10. For example, the scan control function 375 controls a start of a scan, execution of the scan, and end of the scan on the gantry 10 by controlling operations of the X-ray high-voltage apparatus 11, the X-ray detector 13, the gantry control apparatus 16, the data acquisition circuitry 14, and a bed drive apparatus 21.

The display control function 376 perform control to display, on the display 32, images based on various types of image data stored in the memory 35.

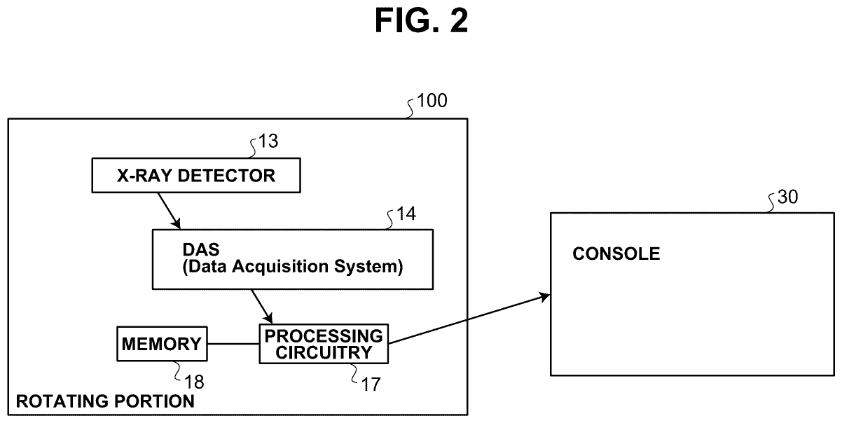

Next, a configuration of a rotating portion 100 according to the first exemplary embodiment will be described with reference to FIG. 2. FIG. 2 illustrates a configuration of the rotating portion 100 according to the first exemplary embodiment. The rotating portion 100 is the rotating part of the X-ray CT apparatus 1 and includes the rotating frame 15 and the X-ray detector 13. The rotating frame 15 is the annular frame that supports the X-ray generation apparatus 12 and the X-ray detector 13 so that the X-ray generation apparatus 12 and the X-ray detector 13 face each other across the subject P, and is rotated at high speed on a circular path centered on the subject P. In addition to the foregoing components, the rotating portion 100 according to the present exemplary embodiment includes the data acquisition circuitry 14, processing circuitry 17, and a memory 18 in order to reduce the amount of data transmission from the rotating portion 100 to a fixed portion (i.e., a fixed part of the X-ray CT apparatus 1, such as the gantry 10) of the X-ray CT apparatus 1 as described below. For example, the X-ray detector 13, the data acquisition circuitry 14, the processing circuitry 17, and the memory 18 are placed in the rotating frame 15.

The memory 18 is implemented using, for example, a semiconductor memory device such as a RAM and a flash memory, a hard disk, or an optical disk, and stores various types of data. The memory 18 is an example of the storage unit.

In the first exemplary embodiment, processing functions executed by the processing circuitry 17 are stored in the forms of computer-executable programs in the memory 18. The processing circuitry 17 is a processor that implements a function by reading a program corresponding to the function from the memory 18 and executing the read program. In other words, the processing circuitry 17 having read the programs has the corresponding functions. The processing circuitry 17 is an example of a 1st processing unit.

The term “processor” used in the description above refers to circuitry such as a CPU, a graphical processing unit (GPU), an application specific integrated circuit (ASIC), a programmable logic device (e.g., a simple programmable logic device (SPLD), a complex programmable logic device (CPLD), or a field programmable gate array (FPGA)). The processor implements the functions by reading the programs stored in the memory 18 and executing the read programs.

While the case where the reconstruction processing function 373, which is an image reconstruction function, in FIG. 1 is included in the processing circuitry 37 of the console 30 is described, the exemplary embodiment is not limited to this case. A reconstruction processing unit may be placed in the fixed portion of the X-ray CT apparatus 1, or as another example, the processing circuitry 17 of the rotating portion 100 may have the reconstruction processing function 373.

Next, the background of the present exemplary embodiment will be described.

In the photon counting X-ray CT apparatus 1, the X-ray detector 13 counts the number of incident X-ray photons within a set energy range using counters referred to as energy bins. The counted data is transmitted from a detector unit of the rotating portion 100 to the fixed portion, and the console 30 performs data preprocessing and image reconstruction as necessary.

Increasing the number of the energy bins improves accuracy of energy information, which is a feature of photon counting X-ray CT apparatuses. However, as the number of the energy bins increases, it becomes difficult to transmit all data in real time due to constraints on the amount of data in transmission of the data from the rotating portion 100 to the fixed portion of the gantry 10.

Based on the foregoing background, the X-ray CT apparatus 1 according to the present exemplary embodiment includes the X-ray detector 13, the data acquisition circuitry 14, the rotating portion 100, the processing circuitry 17 as a processing unit, and the reconstruction processing function 373 as a reconstruction processing unit. The X-ray detector 13 outputs output data corresponding to energy of the X-ray photon emitted from the X-ray tube 12a. The data acquisition circuitry 14 acquires the output data from the X-ray detector 13 and outputs count data for each of the plurality of energy bins. The rotating portion 100 rotatably supports the detector and the data acquisition unit. The processing circuitry 17 is provided in the rotating portion 100 and generates data related to a plurality of material compositions based on the count data.

This enables real-time data transmission from the rotating portion 100 to the fixed portion while utilizing more energy bin data.

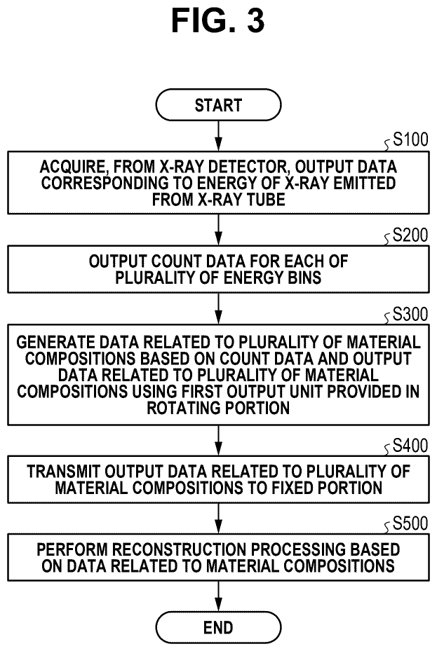

Next, processing according to the present exemplary embodiment will be described with reference to FIGS. 3 to 5. FIG. 3 is a diagram illustrating a flow of processing performed by the X-ray CT apparatus 1 according to the first exemplary embodiment.

First, in step S100, the X-ray detector 13 outputs, to the data acquisition circuitry 14, output data corresponding to the energy of the X-ray photon emitted from the X-ray tube 12a.

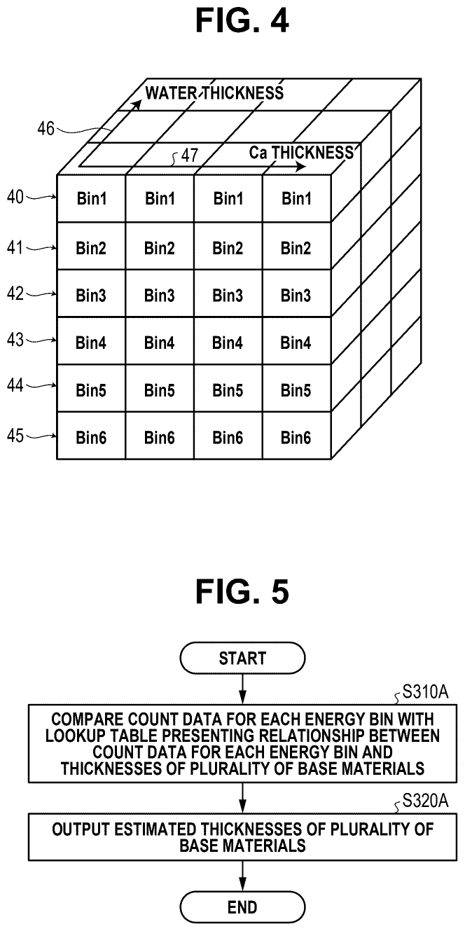

Next, in step S200, the data acquisition circuitry 14 acquires the output data from the X-ray detector 13 and outputs count data for each of the plurality of energy bins. FIG. 4 schematically illustrates count data output from the data acquisition circuitry 14. The data acquisition circuitry 14 acquires the output data from the X-ray detector 13 and outputs, for example, count data 40 to 45 for six energy bins Bin 1 to Bin 6, which correspond to the plurality of energy bins.

Next, in step S300, the processing circuitry 17 provided in the rotating portion 100, which rotatably supports the X-ray detector 13 and the data acquisition circuitry 14, generates data related to the plurality of material compositions (base materials) based on the count data 40 to 45 output in step S200 to output the data related to the plurality of material compositions. The data related to the plurality of material compositions herein refers to, for example, thickness data for the plurality of materials. An example is that the plurality of materials includes water and calcium. Another example is that the plurality of materials includes water, calcium, and an iodinated contrast agent. Yet another example is that the plurality of materials may include a contrast agent other than the iodinated contrast agent and a calculus.

Specifically, the processing circuitry 17 calculates, for example, data 47 representing the thickness of calcium and data 46 representing the thickness of water (Water), based on the count data 40 to 45 output in step S200 as data related to the plurality of material compositions.

FIG. 5 illustrates details of such a process. FIG. 5 is a flowchart illustrating the process in step S300 in FIG. 3 according to the first exemplary embodiment in more detail.

In step S310A, the processing circuitry 17 acquires, from the memory 18 connected to the processing circuitry 17, a lookup table presenting the relationship between the count data for each of the energy bins and the thicknesses of the plurality of base materials. Next, the processing circuitry 17 compares the lookup table presenting the relationship between the count data for each of the energy bins and the thicknesses of the plurality of base materials with the count data for each of the energy bins acquired in step S200.

The lookup table presenting the relationship between the count data for each of the energy bins and the thicknesses of the plurality of base materials is generated, for example, as follows.

In the case where the plurality of base materials is water and calcium, count data is expressed by formula (1) below:

$$\int w ( E ) R ( E ) {exp}^{- j {μ}_{i} ( E ) {d}_{i} di} dE ∼ \int w ( E ) R ( E ) {exp}^{- {μ}_{ca} ( E ) {d}_{ca} - {μ}_{water} ( E ) {d}_{water}} dE . \qquad ( 1 )$$

In formula (1), E denotes the energy, w(E) denotes the energy spectrum of incident X-rays, R(E) denotes the energy response of the detector, denotes the linear attenuation coefficient (μ_{Ca} denotes the linear attenuation coefficient of calcium, and μ_{water} denotes the linear attenuation coefficient of water), and d denotes the transmission distance (d_{Ca} denotes the transmission distance for calcium, d_{water} denotes the transmission distance for water). If the transmission distances of calcium and water are known, the processing circuitry 17 can calculate the count data for each energy bin by substituting the values into the right-hand side of formula (1).

The flow of generating the lookup table is as follows. Prior to scanning, the processing circuitry 17 determines possible combinations of reference materials and thicknesses of the reference materials in advance, and generates an energy bin output lookup table for the reference materials and the thicknesses of the reference materials. Since it is sufficient to know the relative intensity of output for each of the energy bins, a lookup table from which an output ratio can be calculated for each of the energy bins is sufficient. The processing circuitry 17 stores the generated lookup table in the memory 18 connected to the processing circuitry 17.

Next, in step S320A, the processing circuitry 17 outputs the estimated thicknesses of the plurality of base materials based on the comparison results from step S310A. An example is that the processing circuitry 17 selects the thicknesses of the plurality of base materials corresponding to data, in the lookup table, with the count data for the energy bin being closest to the count data for each of the energy bins acquired in step S200, as the estimated thicknesses of the plurality of base materials that are output in step S320A. Further, another example is that the processing circuitry 17 estimates values of the plurality of base materials output in step S320A by selecting a plurality of pieces of data, in the lookup table, being close to the count data for each of the energy bins acquired in step S200, and interpolating the thickness data for the base materials in the pieces of data. As described above, the processing circuitry 17 generates the data related to the plurality of material compositions by referring to the lookup table presenting the relationship between the count data 40 to 45 for each of the energy bins and the thickness data for the plurality of base materials, and performing an interpolation process.

While the case where examples of the plurality of base materials are two types of base materials that are water and calcium is described above, the present exemplary embodiment is not limited to the case, and the plurality of base materials may be three or more types of base materials. For example, the plurality of base materials may be water, calcium, and iodine that is an element associated with contrast agents.

Description is again provided with reference to FIG. 3. In step S400, the processing circuitry 17 transmits the data related to the plurality of material compositions that is output in step S300 from the rotating portion 100 to the fixed portion, or transmits the data to the console 30 through the fixed portion. The data related to the plurality of material compositions that is output in step S300 is smaller in amount than the count data for each of the energy bins that is acquired in step S200. For example, in the case illustrated in FIG. 4, since there are six energy bins in FIG. 4, the number of pieces of data acquired in step S200 is six. On the other hand, two pieces of data, which are water thickness data and calcium thickness data, are output in step S300. Therefore, in this example, the amount of data transmitted is one-third of original data. Accordingly, a reduction in the amount of data transmitted from the rotating portion 100 to the fixed portion or the like is achieved in the X-ray CT apparatus 1.

Next, in step S500, the processing circuitry 37 performs the reconstruction processing using the reconstruction processing function 373 to generate a reconstruction image based on the data related to the plurality of material compositions that is transmitted in step S400. An example is that the processing circuitry 37 placed in the console 30 of the X-ray CT apparatus 1 generates the reconstruction image by, for example, the filtered back projection method or the iterative reconstruction method, using the reconstruction processing function 373 as a reconstruction unit. Another example is that the processing circuitry 37 generates a plurality of reference material images as the reconstruction image using the reconstruction processing function 373. Yet another example is that the processing circuitry 37 generates a reference material image related to water and a reference material image related to calcium as the reconstruction image using the reconstruction processing function 373.

While the case where the reconstruction unit is placed in the console 30 of the X-ray CT apparatus 1 is described above, the present exemplary embodiment is not limited to the case. The reconstruction unit may be placed in the fixed portion of the X-ray CT apparatus 1. In such a case, the reconstruction unit placed in the fixed portion of the X-ray CT apparatus 1 performs the reconstruction processing to generate the reconstruction image based on the data related to the plurality of material compositions that is transmitted in step S400, and thereafter transfers the generated reconstruction image to the console 30.

As described above, the first exemplary embodiment describes the case where the processing circuitry 17 of the rotating portion 100 of the X-ray CT apparatus 1 generates the data related to the plurality of material compositions based on the count data for each of the energy bins by referring to the lookup table. This achieves a reduction in the amount of data transmitted from the rotating portion 100 to the fixed portion.

### Second Exemplary Embodiment

The first exemplary embodiment describes the case where the processing circuitry 17 of the rotating portion 100 of the X-ray CT apparatus 1 generates the data related to the plurality of material compositions by referring to the lookup table. However, the exemplary embodiment is not limited to the case. The processing circuitry 17 of the rotating portion 100 of the X-ray CT apparatus 1 may generate the data related to the plurality of material compositions using a neural network (artificial intelligence (AI)) in step S300.

Specifically, during a learning phase, training of the neural network is performed with the count data for each of the energy bins as input and the thickness data for the plurality of base materials as output. The training of the neural network does not have to be performed by the processing circuitry 17 of the rotating portion 100 and is typically performed by predefined processing circuitry, e.g., the processing circuitry 37 of the console 30 or external processing circuitry. Once the training of the neural network is completed, the processing circuitry that has performed the training transmits the trained neural network to the processing circuitry 17. The processing circuitry 17 stores the trained neural network in the memory 18 of the rotating portion 100.

In step S300, the processing circuitry 17 of the rotating portion 100 acquires the trained model from the memory 18. Next, the processing unit generates the data related to the plurality of material compositions using the trained neural network trained with the count data for each of the energy bins as input and the thickness data for the plurality of base materials as output. In step S400, the processing circuitry 17 transmits the data related to the plurality of material compositions to the fixed portion. Then, in step S500, the processing circuitry 37 performs the reconstruction processing to generate a reconstruction image based on the data related to the plurality of material compositions that is transmitted in step S400 using the reconstruction processing function 373. An example is that the processing circuitry 37 generates a plurality of reference material images as the reconstruction image using the reconstruction processing function 373. Another example is that the processing circuitry 37 generates a reference material image related to water and a reference material image related to calcium as the reconstruction image using the reconstruction processing function 373.

While the present exemplary embodiment describes the case where the data related to the plurality of material compositions is generated using the neural network, the present exemplary embodiment is not limited to the case, and the processing circuitry 17 may generate the data related to the plurality of material compositions using machine learning. For example, the processing circuitry 17 may generate the data related to the plurality of material compositions using principal component analysis (PCA), linear regression, decision trees, and gradient boosting. Further, in the second exemplary embodiment, the data related to the plurality of material compositions may be data on three or more types of materials as in the first exemplary embodiment.

As described above, the second exemplary embodiment describes the case where the processing circuitry 17 generates the data related to the plurality of material compositions using the neural network or machine learning. This achieves a reduction in the amount of data transmitted from the rotating portion 100 to the fixed portion more flexibly.

### Third Exemplary Embodiment

The first and second exemplary embodiments describe the case where the thicknesses of the reference materials are calculated as the data related to the plurality of material compositions. However, the exemplary embodiments are not limited to the case. A third exemplary embodiment describes a case where effective atomic numbers are calculated as the data related to the plurality of material compositions, instead of the thicknesses of the reference materials.

In the third exemplary embodiment, processes similar to those in the first exemplary embodiment are performed, except for the process in step S300 in FIG. 3. Redundant descriptions of the processes other than the process in step S300 are omitted herein.

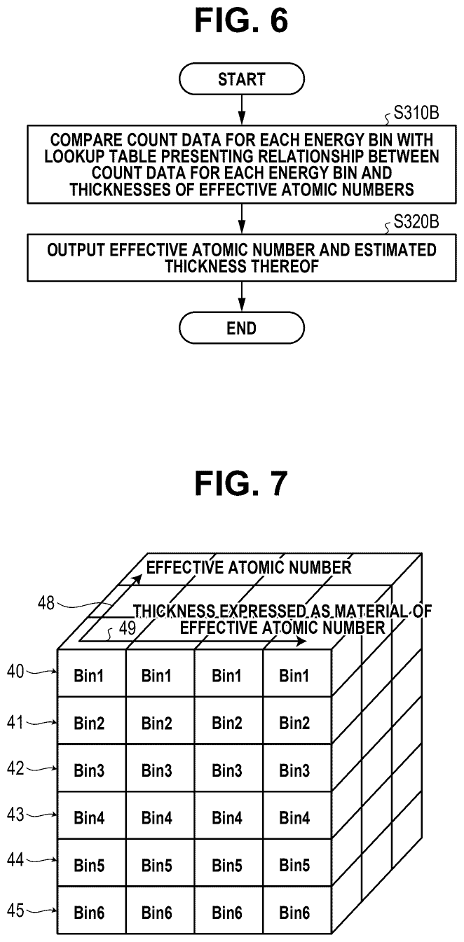

FIG. 6 illustrates the process in step S300 in FIG. 3 in the third exemplary embodiment in detail.

In step S310B, the processing circuitry 17 compares the count data for each of the energy bins with a lookup table from the memory 18 connected to the processing circuitry 17. The lookup table presents the relationship between the count data for each of the energy bins and the effective atomic numbers and their thicknesses, i.e., the thicknesses expressed as the materials with the effective atomic numbers.

The flow of generating the lookup table is as follows. Prior to imaging, the processing circuitry 17 determines possible combinations of effective atomic numbers and their thicknesses in advance, and generates an energy bin output lookup table for the effective atomic numbers and their thicknesses. The processing circuitry 17 stores the generated lookup table in the memory 18 connected to the processing circuitry 17.

Next, in step S320B, the processing circuitry 17 outputs the estimated thicknesses of the plurality of base materials based on the comparison results from step S310B. Specifically, the processing circuitry 17 generates the effective atomic numbers and their estimated thicknesses as the data related to the plurality of material compositions. Specifically, in the third exemplary embodiment, the data related to the plurality of material compositions is data including an effective atomic number. For example, by referring to the lookup table presenting the relationship between the count data 40 to 45 for each of the energy bins and the effective atomic numbers and their thicknesses, the processing circuitry 17 generates effective atomic numbers 48 and thicknesses 49 expressed as the materials of the effective atomic numbers 48 as the data related to the plurality of material compositions, as illustrated in FIG. 7. Based on the generated data of the effective atomic numbers 48 and the thicknesses 49, the processing circuitry 37 can generate a reference material image.

As described above, in the third exemplary embodiment, the processing circuitry 17 generates data including the effective atomic numbers as the data related to the plurality of material compositions. This achieves a reduction in the amount of data transmitted from the rotating portion 100 to the fixed portion in a manner different from the first and second exemplary embodiments.

### Fourth Exemplary Embodiment

The first and second exemplary embodiments describe the case where the thicknesses of the plurality of materials are used as the data related to the plurality of material compositions, and the third exemplary embodiment describes the case where the effective atomic numbers are used as the data related to the plurality of material compositions. A fourth exemplary embodiment describes a case where photoelectric effect data, Compton scattering data, and data corresponding to a specified tube voltage are used as the data related to the plurality of material compositions.

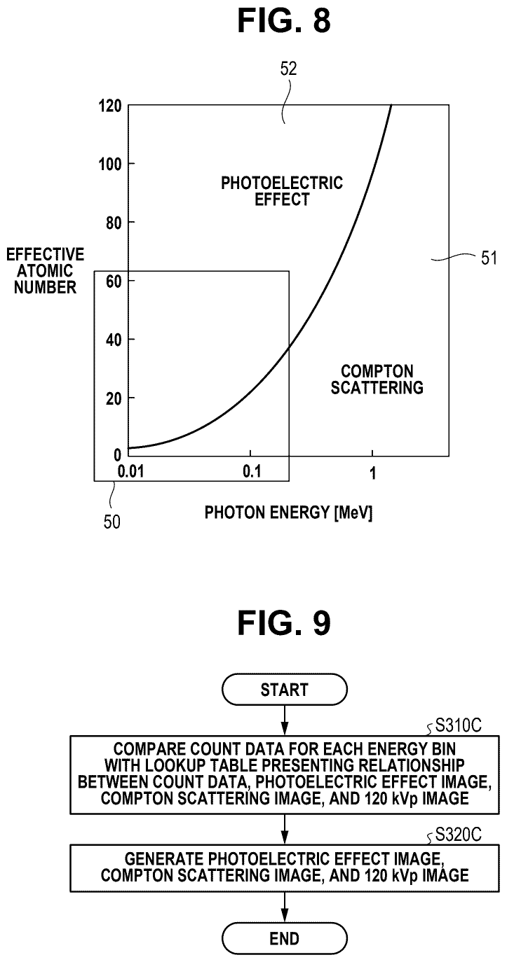

The meaning of using the photoelectric effect data and the Compton scattering data as the data related to the plurality of material compositions will be described below with reference to FIG. 8. FIG. 8 is a schematic diagram illustrating which process becomes dominant in each region, with the horizontal axis representing the photon energy and the vertical axis representing the effective atomic number. In FIG. 8, a region 50 is a region that is typically used in X-ray CT. From FIG. 8, it can be observed that a photoelectric effect becomes dominant in a region 52 whereas Compton scattering is dominant in a region 51. Further, it can be observed that as the value of the effective atomic number changes, the ratio between the photoelectric effect and Compton scattering in the count data varies. Thus, the processing circuitry 17 calculates data (photoelectric effect data or photoelectric effect image) corresponding to the frequency at which the photoelectric effect occurs and data (Compton scattering data or Compton scattering image) corresponding to the frequency at which Compton scattering occurs. In addition to the foregoing data, the processing circuitry 17 calculates data corresponding to a specified tube voltage, e.g., 120 kVp image data, which corresponds to data acquired by a normal integrating detector, and transmits the three pieces of data to the fixed portion. In a case where the number of energy bins is six, for example, the number of pieces of data transmitted to the fixed portion is three, achieving a reduction in the amount of data transmitted from the rotating portion 100 to the fixed portion.

FIG. 9 illustrates a process performed by the X-ray CT apparatus 1 in step S300 in FIG. 3 according to the fourth exemplary embodiment. The processes other than the process in step S300 are similar to those in the first exemplary embodiment, so that redundant descriptions of the processes other than the process in step S300 are omitted herein.

In step S310C, the processing circuitry 17 acquires, from the memory 18 connected to the processing circuitry 17, the count data for each of the energy bins and a lookup table presenting the relationship between the count data for each of the energy bins, the photoelectric effect data (photoelectric effect image), the Compton scattering data (Compton scattering image), and the data corresponding to the specified tube voltage (e.g., 120 kVp image). Next, the processing circuitry 17 acquires the count data for each of the energy bins. Then, the processing circuitry 17 compares the lookup table with the count data for each of the energy bins acquired in step S200.

The flow of generating the lookup table is as follows. Prior to imaging, the processing circuitry 17 converts the count data for each of the energy bins into the data corresponding to the frequency at which the photoelectric effect occurs and the data corresponding to the frequency at which Compton scattering occurs based on, for example, a theoretical curve illustrated in FIG. 8, for possible combinations of count data for each of the energy bins in advance. The processing circuitry 17 also converts the count data for each of the energy bins into the data corresponding to the specified tube voltage, e.g., 120 kVp image. As described above, the processing circuitry 17 generates the lookup table and stores the generated lookup table in the memory 18 connected to the processing circuitry 17.

Next, in step S320C, the processing circuitry 17 generates the photoelectric effect data (photoelectric effect image), the Compton scattering data (Compton scattering image), and the data corresponding to the specified tube voltage (120 kVp image) based on the comparison results from step S310C and transmits the generated data to the fixed portion. The processing circuitry 37 reconstructs an X-ray CT image based on the transmitted data using the reconstruction processing function 373. The processing circuitry 37 can generate a reference material image based on the photoelectric effect image, the Compton scattering image, and the 120 kVp image.

The foregoing exemplary embodiment describes the case where the processing circuitry 17 outputs the data related to the plurality of material compositions for each X-ray detector 13 and transmits the data to the fixed portion. However, the exemplary embodiment is not limited to the case. For example, the processing circuitry 17 of the rotating portion 100 may have an image reconstruction function, aggregate data acquired from the plurality of X-ray detectors 13, perform image reconstruction to generate a photoelectric effect image, a Compton scattering image, and a 120 kVp image, and transmit the generated images to the fixed portion.

At least one of the exemplary embodiments described above achieves a reduction in the amount of data transferred.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An X-ray computed tomography (CT) apparatus comprising:
an X-ray detector configured to output output data corresponding to energy of an X-ray photon;
a data acquisition unit configured to acquire the output data from the X-ray detector and output count data for each of a plurality of energy bins;
a rotating portion configured to rotatably support the X-ray detector and the data acquisition unit;
a 1st processing circuitry provided in the rotating portion and configured to generate data related to a plurality of material compositions based on the count data and output the data related to the plurality of material compositions; and
a 2nd processing circuitry configured to perform a reconstruction process based on the data related to the plurality of material compositions.

2. The X-ray CT apparatus according to claim 1, wherein the 2nd processing circuitry is placed in a fixed portion of the X-ray CT apparatus.

3. The X-ray CT apparatus according to claim 1, wherein the 2nd processing circuitry is placed in a console of the X-ray CT apparatus.

4. The X-ray CT apparatus according to claim 1, wherein the data related to the plurality of material compositions includes thickness data for a plurality of materials.

5. The X-ray CT apparatus according to claim 4, wherein the plurality of materials is water and calcium.

6. The X-ray CT apparatus according to claim 4, wherein the plurality of materials is water, calcium, and an iodine contrast agent.

7. The X-ray CT apparatus according to claim 1, wherein the 1st processing circuitry is configured to generate the data related to the plurality of material compositions by referring to a lookup table presenting a relationship between the count data for each of the energy bins and thickness data for a plurality of base materials and performing an interpolation process.

8. The X-ray CT apparatus according to claim 1, wherein the 1st processing circuitry is configured to generate the data related to the plurality of material compositions using a trained neural network trained with the count data for each of the energy bins as input and thickness data for a plurality of base materials as output.

9. The X-ray CT apparatus according to claim 1, wherein the data related to the plurality of material compositions is data including an effective atomic number.

10. The X-ray CT apparatus according to claim 1, wherein the data related to the plurality of material compositions includes photoelectric effect data, Compton scattering data, and data corresponding to a specified tube voltage.
